(19) United States
(12) Patent Application Publication
(10) Pub. No.: US 2020/0025737 A1
(43) Pub. Date: Jan. 23, 2020

(54) CHEMICAL SENSOR DATA RECOGNITION

(71) Applicant: KING FAHD UNIVERSITY OF PETROLEUM AND MINERALS, Dhahran (SA)

(72) Inventors: Basem AL MADANI, Dhahran (SA); Sadam Hussein Al-Azani, Dhahran (SA)

(73) Assignee: KING FAHD UNIVERSITY OF PETROLEUM AND MINERALS, Dhahran (SA)

(21) Appl. No.: 16/263,865

(22) Filed: Jan. 31, 2019

Related U.S. Application Data
(60) Provisional application No. 62/276,582, filed on Jan. 8, 2016.

(51) Int. Cl. G01N 33/00

(52) U.S. Cl. CPC G01N 33/0073

(57) ABSTRACT

Methods, systems, and computer readable media for chemical sensor data recognition using an odor detection system with sensor modules.

### CROSS REFERENCE TO RELATED APPLICATIONS

This application claims the benefit of U.S. Provisional Application No. 62/276,582 filed. Jan. 8, 2016, which is incorporated herein by reference in its entirety.

### BACKGROUND

### Technical Field

The present disclosure is directed generally to computer based sensing, and, more particularly, to methods, computer readable media, and systems for chemical sensor data recognition.

### Description of Related Art

Developing electronic nose (e-nose) systems and olfactory machines has received significant interest. Such systems may have a crucial role in the development of various real life applications in civil and industrial environments such as finding drugs and explosives, quality control in food processing, detection and diagnosis of illness and the estimation of blood alcohol content (BAC) for drivers (see F.-M. Schleif, B. Hammer, J. G. Monroy, J. G. Jimenez, J.-L. Blanco-Claraco, M. Biehl, and N. Petkov, “Odor recognition in robotics applications by discriminative time-series modeling,” Pattern Anal. Appl., pp. 1-14, 2015, which is incorporated herein by reference).

Another important application includes robots equipped with chemical sensors in which adaptive and biologically inspired strategies may be effective. Basic steps of odor recognition can include (i) signal conditioning and feature extraction, (ii) feature selection and (iii) classification.

Feature selection aims at reducing the “curse of dimensionality” of a given dataset by selecting the most significant attributes and eliminating the redundant, irrelevant, and/or noisy features for several tasks including classification, clustering, navigation or diagnostics.

Feature selection is becoming an important data processing step before applying machine learning to build robustness in classification models and to help overcome over-fitting problem (see M. A. Hall and L. A. Smith, “Feature Selection for Machine Learning: Comparing a Correlation-Based Filter Approach to the Wrapper.,” in FLAIRS conference, 1999, vol. 1999, pp. 235-239, which is incorporated herein by reference). There are three fundamental approaches of feature selection techniques namely: filter, wrapper, and embedded methods. The filter approach relies on general characteristics of the data to make an independent assessment including filtering the attributes to select the most important subsets, hence the technique is called filter method.

On the other hand, the wrapper approach employs machine learning methods to evaluate the subset of features. It is called wrapper because a learning algorithm is wrapped into the selection procedure. The third approach embeds the feature selection and training process, hence it is called embedded method. For example, filter methods and wrapper methods have been used to select the best subset of features from chemical sensor dataset (see X. R. Wang, J. T. Lizier, T. Nowotny, A. Z. Berea, M. Prokopenko, and S. C. Trowell, “Feature selection for chemical sensor arrays using mutual information,” PLoS One, vol. 9, no. 3, p. e89840, 2014; A. Krause, A. Singh, and C. Guestrin, “Near-optimal sensor placements in Gaussian processes: Theory, efficient algorithms and empirical studies,” J. Mach. Learn. Res., vol. 9, pp. 235-284, 2008; T. Nowotny, A. Z. Berna, R. Binions, and S. Trowell, “Optimal feature selection for classifying a large set of chemicals using metal oxide sensors,” Sensors Actuators B Chem., vol. 187, pp. 471-480, 2013; and M. Pardo and G. Sberveglieri, “Comparing the performance of different features in sensor arrays,” Sensors Actuators B Chem., vol. 123, no. 1, pp. 437-443, 2007, each of which is incorporated herein by reference).

Several machine learning techniques have been used to classify chemicals including SVM, multilayer perceptrons (MLP), Bayesian networks, k-NN, etc.

The curse of dimensionality is one of the major problems of sensor array datasets. Researchers have investigated the effects of applying feature selection techniques to improve the performance of classification methods. Nowotny et al. used wrapper feature selection to determine the most significant set of features with the best SVM based classification performance (see T. Nowotny, A. Z. Berra, R. Binions, and S. Trowell, “Optimal feature selection for classifying a large set of chemicals using metal oxide sensors,” Sensors Actuators B Chem., vol. 187, pp. 471-480, 2013, which is incorporated herein by reference). A set of 20 chemicals using two types of sensors, classical doped tin oxide and zeolite-coated chromium titanium oxide sensors was used. It is reported that the performance of the selected subset of features was better than the complete features. However, since wrapper methods require executing learning and inference algorithms on all possible combinations of features, they might be more expensive in term of running time compared with filter methods. In this context, Wang et al. evaluated filter feature selection methods (mutual information) to select the most significant set of features from the same data set used by Nowotny and compared the performance of both methods (see X. R. Wang, J. T. Lizier, T. Nowotny, A. Z. Berra, M. Prokopenko, and S. C. Trowell, “Feature selection for chemical sensor arrays using mutual information,” PLoS One, vol. 9, no. 3, p. e89840, 2014, which is incorporated herein by reference). It was reported that although wrapper methods conducts exhaustive search of all permitted feature combinations, the selected features are closely matched. Wang et al. also evaluated the powerful of the selected features using several classifiers namely, Bayesian networks, k-MN, neural networks, mutual information maximum likelihood in addition to SVM and it is reported that Bayesian networks achieved the highest recognition rates. (See, X. R. Wang, J. T. Liner, T. Nowotny, A. Z. Berm, M. Prokopenko, and S. C. Trowell, “Feature selection for chemical sensor arrays using mutual information,” PLoS One, vol. 9, no. 3, p. e89840, 2014, which is incorporated herein by reference).

Chen et al. introduced an approach to detect three types of gases (CO, H2 and CH4) and their concentrations using BP feed forward neural network (see J. Chen, L. He, Y. Quart, and W. Jiang, “Application of BP Neural Networks based on genetic simulated annealing algorithm for short term electricity price forecasting,” in Advances in Electrical Engineering (ICAEE), 2014 International Conference on, 2014, pp. 1-6, which is incorporated herein by reference). The Chen et al. approach was evaluated using a synthetic dataset.

Schleif et al. extended classical generative topographic mapping through time (GTM-TT) by integrating supervised classification and relevance learning to classify odor (see F.-M. Schleif, B. Hammer, J. G. Monroy, J. G. Jimenez, J.-L. Blanco-Claraco, M. Biehl, and N. Petkov, “Odor recognition in robotics applications by discriminative time-series modeling,” Pattern Anal. Appl., pp. 1-14 2015, which is incorporated herein by reference). The Schleif et al. approach was evaluated using an e-nose comprising an array of metal oxide sensors (MOX) to classify samples of seven different volatiles under uncontrolled conditions. The recognition rate of the proposed model was then compared with K-NN, SVM and a reservoir computing time-series kernel (RTK), using four different datasets. The used model, SGTM-TT, outperformed other techniques using one dataset out of four datasets.

Some factors and events might affect the performance of sensors and lead to failures. The effects of these factors ranges from modest signal drift to severe defect. In this context, a slandered dataset for six different volatile organic compounds has been built over a period of three years under tightly controlled operating conditions using an array of 16 metal-oxide gas sensors (see A. Szczurek, B. Krawczyk, and M. Maciejewska, “VOCs classification based on the committee of classifiers coupled with single sensor signals,” Chemom. Intell. Lab. Syst., vol. 125, pp. 1-10, 2013, which is incorporated herein by reference). Ensemble based SVM classifier was applied to solve a gas identification problem. Experiments clearly indicate the presence of drift in the sensors during the period of three years and that it degrades the performance of the classifiers.

Martinelli, Magna, Vergara, & Di Natale introduced an approach to increase the robustness of chemical sensors and alleviate the problems of sensors failure (see E. Martinelli, G. Magna, A. Vergara, and C. Di Natale, “Cooperative classifiers for reconfigurable sensor arrays,” Sensors Actuators B Chem., vol. 199, pp. 83-92, 2014, which is incorporated herein by reference). Their approach is based on cooperation of a set of sub-classifiers (equal to the number of sensors one for each sensor). The final classification decision is made using weighted majority voting decision rule. This model is evaluated using a synthetic and a real gas sensor array with three classes (acetaldehyde, ethylene and toluene). To evaluate the model readability against the fault tolerance property, it is reported that the sensors suffered from drift in case of the real dataset. In case of the synthetic dataset, fault events were induced in the array. It is reported that the Martinelli et al. model outperformed the K-NN classifier.

Miao, Zhang, Wang, and Li conducted research to select an optimal sensor to classify nine kinds of ginsengs with an e-nose system consisting of 12 metal oxide sensors. A linear discriminant analysis classification method was used and it was concluded that as the number of samples increased, the average minimum number of sensors increased, while the increment decreased gradually and the average optimal classification rate decreased gradually. (See, J. Miao, T. Zhang, Y. Wang, and G. Li, “Optimal Sensor Selection for Classifying a Set of Ginsengs Using Metal-Oxide Sensors,” Sensors, vol. 15, no. 7, pp. 16027-16039, 2015, which is incorporated herein by reference).

Accordingly, in light of the above mentioned problems and limitations of conventional programming techniques, methods and tools, a need exists for methods detecting and classifying materials according to sensor input.

### SUMMARY

Some implementations of the method and invention can include and/or combine both filter and wrapper approaches.

Some implementations can include a method comprising receiving sensor data from one or more sensors, and extracting one or more features from the sensor data. The method can also include selecting a group of selected features from among the one or more features, and classifying the group of selected features using one or more models. The method can further include providing an indication of sensed material based on the classifying.

In some implementations, the one or more sensors can include chemical sensors and the sensed data corresponds to data from the chemical sensors. The extracting can include extracting a portion of time series data from the sensor data.

In some implementations, selecting the group of selected features can include performing one or more feature selection techniques to determine one or more significant features, wherein the group of selected features includes the one or more significant features.

In some implementations, classifying can include performing ensemble classification. The selecting can include performing a heterogeneous feature selection technique including one or more of filtering, wrapping, or an embedded technique. The sensed material can include one or more gases and their respective concentrations.

Some implementations can include a non-transitory computer readable medium having instructions stored therein that, when executed by one or more processors, cause the one or more processors to perform a method. The method can include receiving sensor data from one or more sensors, and extracting one or more features from the sensor data. The method can also include selecting a group of selected features from among the one or more features, and classifying the group of selected features using one or more models. The method can further include providing an indication of sensed material based on the classifying.

In some implementations, the one or more sensors can include chemical sensors and the sensed data corresponds to data from the chemical sensors. The extracting can include extracting a portion of time series data from the sensor data.

In some implementations, selecting the group of selected features can include performing one or more feature selection techniques to determine one or more significant features, wherein the group of selected features includes the one or more significant features.

In some implementations, classifying can include performing ensemble classification. The selecting can include performing a heterogeneous feature selection technique including one or more of filtering, wrapping, or an embedded technique. The sensed material can include one or more gases and their respective concentrations.

Some implementations can include a system comprising one or more processors coupled to a non-transitory computer readable medium having stored thereon software instructions that, when executed by the one or more processors, cause the one or more processors to perform operations. The operations can include receiving sensor data from one or more sensors, and extracting one or more features from the sensor data. The operations can also include selecting a group of selected features from among the one or more features, and classifying the group of selected features using one or more models. The operations can further include providing an indication of sensed material based on the classifying.

In some implementations, the one or more sensors can include chemical sensors and the sensed data corresponds to data from the chemical sensors. The extracting can include extracting a portion of time series data from the sensor data.

In some implementations, selecting the group of selected features can include performing one or more feature selection techniques to determine one or more significant features, wherein the group of selected features includes the one or more significant features.

In some implementations, classifying can include performing ensemble classification. The selecting can include performing a heterogeneous feature selection technique including one or more of filtering, wrapping, or an embedded technique. The sensed material can include one or more gases and their respective concentrations.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is a diagram of an example chemical sensor data recognition system in accordance with some implementations.

FIG. 2 is a chart of full and selected features in accordance with some implementations.

FIG. 3 is a diagram showing an example chemical sensor data recognition data flow in accordance with some implementations.

FIG. 4 is a diagram of an example processing device configured for chemical sensor data recognition in accordance with some implementations.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise. The drawings are generally drawn to scale unless specified otherwise or illustrating schematic structures or flowcharts.

Aspects of this disclosure are directed to methods, systems, and computer readable media for computerized chemical sensor data recognition and chemical detection based on an approach described herein.

Some implementations can include a method to improve the accuracy of classifiers using ensemble classification methods. Ensemble classification methods include combining the results of multiple classifiers in order to improve results. There are three fundamental ensemble approaches: bagging, boosting and stacking or blending. Some implementations can include a stacking approach as ensemble classification method as well as common individual classifier namely, k-nearest Neighbor (k-NN), Sequential Minimum Optimization based on Support Vector Machine (SMO-SVM), MLP and Naïve Bayes (NB).

Some implementations address the problem of feature selection for classifying chemical gases with their concentrations (12 classes) using an array of 16 MOX chemical sensors in an electronic nose. Some implementations include using an ensemble classification method to improve the effectiveness of individual classifiers to classify large number of chemicals.

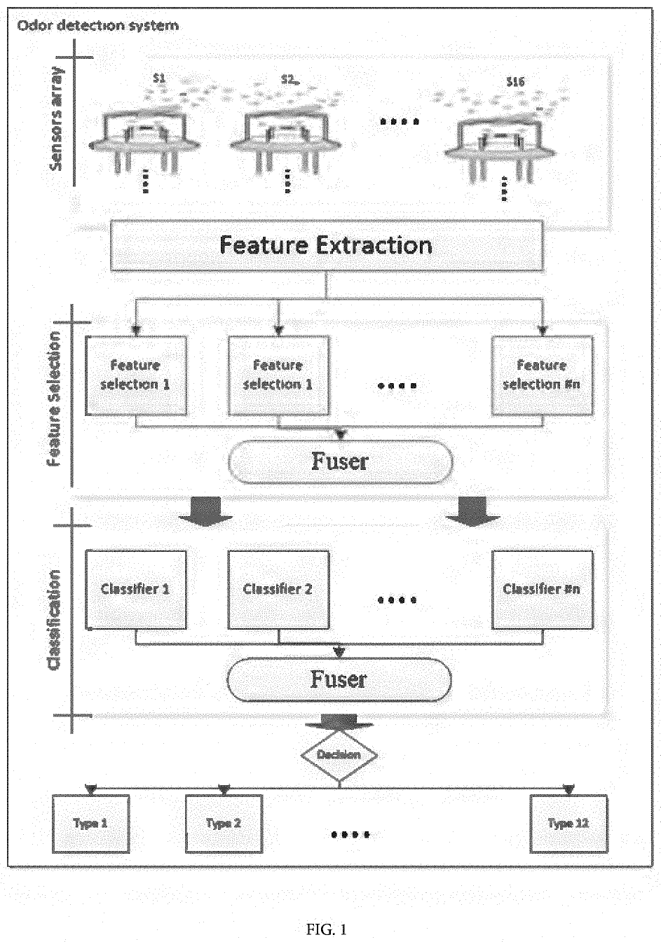

As shown in FIG. 1, some implementations can include several steps, namely feature extraction, feature selection, classification and detection.

### Feature Extraction

To extract features from the row dataset which includes 928 lines (58 samples*16 sensors) with 7500 data points for each measurement per sensor, each 16 consecutive lines are required to be read to obtain a single measurement from 16 time-series of the sensors. (See, A. Ziyatdinov, J. Fonollosa, L. Fernandez, A. Gutierrez-Gàlvez, S. Marco, and A. Perera, “Bioinspired early detection through gas flow modulation in chemo-sensory systems,” Sensors Actuators B Chem., vol. 206, no. 0, pp. 538-547, 2015, which is incorporated herein by reference). These features are extracted and stored in a file (e.g., a comma separated value file) with 58 samples distributed among 12 chemicals by (see http://archive.ics.uci.edu/ml/datasets/Gas+sensor+array+under+flow+modulation, which is incorporated herein by reference). The statistics of the dataset are shown in Table I, below.

TABLE I
THE DATASET STATISTICS

| Name of class | # of samples |
|---|---|
| eth-0.1 | 6 |
| eth-0.3 | 4 |
| eth-1 | 5 |
| ace-0.1 | 6 |
| ace-0.3 | 6 |
| ace-1 | 3 |
| ace-0.1-eth-0.1 | 4 |
| ace-0.1-eth-0.3 | 5 |
| ace-0.3-eth-0.1 | 5 |
| ace-0.1-eth-1 | 3 |
| ace-1-eth-0.1 | 3 |
| air | 8 |

### Feature Selection

Some implementations can include a combination of several feature selection techniques including a Correlation-based Feature Selection (CFS) technique with two different search methods namely Best First (BF) and Rank Search (RS) which are abbreviated as CFS+BF and CFS+RS. Additionally, the techniques can include Chi-square, Information Gain (IG), RelifF and SVMAttributeEval. Chi-square, Information Gain (IG), RelifF and SVMAttributeEval sorts attributes based on their individual evaluations. Some implementations can include different cutoff thresholds of Chi-square, Information Gain (IG), RelifF and SVMAttributeEval instead of the default threshold (−1.8) to help reduce or eliminate attributes with ranks of the defined threshold or lower. Some implementations can include selecting features using single feature selection techniques, and then extracting the best attributes among them. A feature can be defined to be the most significant feature if it will be selected by all feature selection techniques. Three levels can be used to identify the best significant features: the features that are selected by all features selection techniques (voting with 100%), and features selected by five feature selection techniques (voting with 83%) and features selected by four feature selection techniques (voting with 67%).

Chemical types or families that may be used as a feature basis include CO sensors, ozone sensors, sulfide sensors, alcohol sensors, nitrogen oxide sensors, hydrocarbon sensors, volatile organic component sensors, amines, and the like.

### Classification

In classification, several models can be generated individually and in combination. The focus is to utilize ensemble classification methods; however the single classifiers can be used for comparison and as guidance for selecting the ensemble classifiers. As individual classifiers we used k-NN, SMO-SVM, MLP and Naïve Bayes can be used. The selected features in the previous step can be used as input to several individual and ensemble classification methods. For ensemble classification methods, different combinations are possible.

### Detection

The odor detection system preferably has a plurality of sensors configured to detect the presence of one or more chemical compounds or chemical types in an airborne sample. The analysis and/or detection of chemical compounds or chemical types is carried out on a volatile fraction of a sample. Although a sample may be in solid or liquid form, gaseous components may diffuse from or evaporate from the sample. It is the volatile fraction sample that is responsible for typical olfactory sensory input for mammals and is the basis for the detection and feature characterization of the present invention.

In one aspect of the odor detecting in the system (see FIG. 1) a sample chamber that is optionally thermostatted, for example maintained at a particular temperature or subjected to a particular temperature regimen, is connected via one or more tubes that permit passage of a gas stream over the sample and redirection of the gas stream to one or more sensor modules that make up the sensor array of the present odor detection system, with each module containing one or more electrochemical sensors. The sample chamber may also be subject to varying pressure conditions. In one aspect the sample chamber is configured to subject a solid, liquid or gaseous sample to varying pressures, such as varying degrees of vacuum (e.g., 0.01 bar, 0.1 bar, 0.2 bar, 0.5 bar, 0.85, 0.9 bar or about one bar). Alternately the sample chamber may subject a solid, liquid or gaseous sample to a pressure increase by either pressurization with a carrier gas or by heating the sample chamber under closed conditions. The use of different temperature and pressure regimens/conditions for collecting and directing sample gases to the sensor modules results in different feature characteristics and sensitivity towards a different spectrum of detected chemicals or chemical types.

In another aspect of the invention the gaseous sample containing the volatile components, and optionally a carrier gas, passing from the sample chamber to one or more sensor modules may include one or more filters configured for mechanical or chemical filtration. Mechanical filtration may include screens, frits or other porous obstructions whereby solid particulate materials are mechanically removed from the gas stream exiting from the sample chamber prior to entry into a sensor module. In addition, optionally, one or more chemical filters may be installed downstream from the sample chamber to selectively remove chemicals from the gas stream to the sensor module. For example, an activated carbon filter may be used to remove or reduce the concentration of organic materials in the carrier gas stream to thereby increase sensitivity towards non-organic or non-hydrocarbon materials. For example, an activated carbon filter may be used to remove volatile hydrocarbons such as toluene, hexane or other hydrocarbon residues thereby leaving a gas stream that is relatively enriched in inorganic gases or gaseous materials that are otherwise not absorbed by activated carbon. Alternately, the chemical filter may contain a liquid portion through which the carrier gas is bubbled. One or more reactive components in the liquid portion may selectively precipitate or immobilize certain chemical classes or chemical compounds present in the sample gas thereby permitting enrichment of other components and avoidance of sensory overload by one or more components present in the sample gas at excessively high concentration.

Electrochemical sensors may include metal oxide-type sensors, conductive polymer-type sensors, spectrophotometric sensors, gravimetric sensors (e.g., IR, UV, NMR, GC, XPS etc.) gas chromatographic and semiconductor sensors. Preferably the sensor modules contain one or more metal oxide sensors in combination with one or more electrochemical sensors based on semiconductor construction. Gas diffusion into the semiconductor sensor through a porous membrane with or without chemical oxidation or reduction occurring provides a means for detecting and quantifying through an electrochemical signal the responsiveness or concentration of one or more chemical compounds.

Preferably each module contains at least one chemical sensor, preferably a combination of a metal oxide semiconductor sensor, in a single easily removable module that may be connected to the sample gas stream in series or in parallel with one or more other sensor modules. The sensor module may separately be sealed or maintained under particular pressure conditions to enhance responsiveness to one or more types of chemicals.

### Gas Type and Concentration Detection

The generated models in the previous step can then be used in this step to identify a plurality of classes, functionalities and/or types of chemicals.

Chemicals may be broadly detected/defined according to type (such as alkanes, alkenes, hydrocarbons, alcohols, amines, aldehydes, ketones, carbonyl-containing, inorganic, halogenated, otherwise substituted, and the like). Chemical types may be classified and characterized cumulatively or in specific subgroups. For example, chemicals containing a carbonyl group may be detected by one or more sensors including infrared detection capabilities. The feature derived from measurements of the sample gas may include one or more major peaks in the infrared spectrum. Peaks within a certain range may be cumulatively used as representation of the total amount of carbonyl-containing components of chemical compounds present in a sample gas stream (for carbonyl, as an example, sharp peaks typically within the region of 1,500-2,000 cm⁻¹). Alternately, or in addition, the particular peak positions of individual compounds or sub-types of carbonyl group-containing molecules may be used as a basis for correlating the amounts of particular chemical species or chemical groups present in a gas sample mixture. For example, a carbonyl group having a peak at about 1715 cm⁻¹ may correlate with the presence of a carbonyl group-containing chemical compound such as dimethyl ketone. Therefore, the features of a particular sensor such as infrared or the voltage signal from a metal oxide or semiconductor sensor can be used as a basis for characterizing particular types of chemical compounds and/or particular classes or species of chemical compounds. One source of electrochemical sensors useful for the present invention is SPEC Sensors of Newark, Calif.

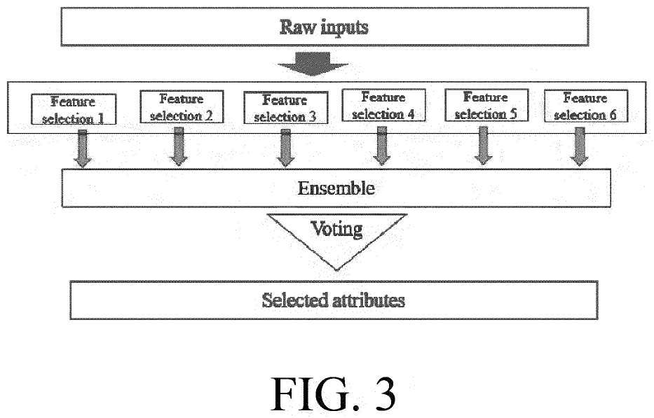

Some implementations can include an ensemble of feature selection techniques based on voting. Conventional systems may apply a single feature selection technique to select the most important features or attributes. However, implementations of the disclosed subject matter can include a technique to select the most important features/attributes based on the voting ensemble to detect different chemical gases and their respective concentrations. Some implementations can be based on several single feature selection techniques namely: Correlation-based Feature Selection technique with two different search methods namely Best First and Rank Search, Chi-square, Information Gain, RelifF and SVM-based feature selection (see FIG. 3).

Some implementations can include a heterogeneous feature selection technique that combines one or more main types of feature selection namely: filtering, wrapping and embedded methods in parallel. The final decision can be made based on voting, which may be more efficient than single feature selection techniques individually as reported in the conducted experimental results. Some implementations can result in selecting a very small number of attributes which are more significant. Since the single feature selection techniques work in parallel (not in sequence) they do not require a large execution time and can accelerate the training time. This can help avoid the issue of overfitting caused by the curse of dimensionality (high-dimensional spaces) and can generate more efficient machine learning models. Additionally, an accurate system can be developed to detect gases and their concentrations. An implementation of the disclosed system was evaluated using several classification methods and 100% results in terms of precision, recall, F1 and accuracy are obtained many times using small number of attributes selected using the disclosed technique.

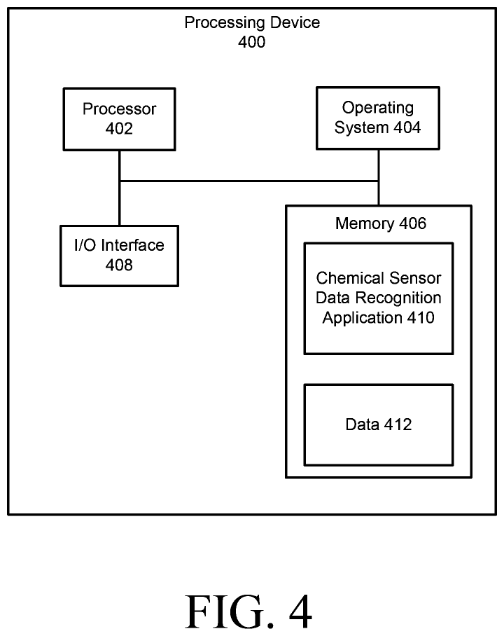

FIG. 4 is a block diagram of an example processing device 400 which may be used to implement one or more features described herein. In one example, device 400 may be used to implement a computer device including an authentication system (e.g., 102), and perform appropriate method implementations described herein (e.g., one or more of the steps shown in FIGS. 1 and 3). Device 400 can be any suitable computer system, server, or other electronic or hardware device. For example, the device 400 can be a mainframe computer, desktop computer, workstation, portable computer, or electronic device (portable device, mobile device, cell phone, smart phone, tablet computer, television, TV set top box, personal digital assistant (PDA), media player, game device, wearable device, etc.). In some implementations, device 400 includes a processor 402, an operating system 404, a memory 406, and input/output (I/O) interface 408.

Processor 402 can be one or more processors and/or processing circuits to execute program code and control basic operations of the device 400. A “processor” includes any suitable hardware and/or software system, mechanism or component that processes data, signals or other information. A processor may include a system with a general-purpose central processing unit (CPU), multiple processing units, dedicated circuitry for achieving functionality, or other systems. Processing need not be limited to a particular geographic location, or have temporal limitations. For example, a processor may perform its functions in “real-time,” “offline,” in a “batch mode,” etc. Portions of processing may be performed at different times and at different locations, by different (or the same) processing systems. A computer may be any processor in communication with a memory.

Memory 406 is typically provided in device 400 for access by the processor 402, and may be any suitable processor-readable storage medium, e.g., random access memory (RAM), read-only memory (ROM), Electrical Erasable Read-only Memory (EEPROM), Flash memory, etc., suitable for storing instructions for execution by the processor, and located separate from processor 402 and/or integrated therewith. Memory 406 can store software operating on the device 400 by the processor 402, including an operating system 404, one or more applications 410, and associated data 412. In some implementations, applications 410 can include instructions that enable processor 402 to perform the functions described herein, e.g., some or all of the methods of FIGS. 1 and 3.

For example, applications 410 can include a chemical sensor data recognition application as described herein. Any of software in memory 404 can alternatively be stored on any other suitable storage location or computer-readable medium. Memory 404 and any other type of storage (magnetic disk, optical disk, magnetic tape, or other tangible media) can be considered “storage” or “storage devices.”

I/O interface 408 can provide functions to enable interfacing the processing device 400 with other systems and devices. For example, chemical sensors, network communication devices, storage devices (e.g., memory and/or database), and input/output devices can communicate via interface 408. In some implementations, the I/O interface 408 can connect to interface devices including input devices (chemical sensors, keyboard, pointing device, touchscreen, microphone, camera, scanner, etc.) and/or output devices (display device, speaker devices, printer, motor, etc.).

For ease of illustration, FIG. 4 shows one block for each of processor 402, memory 406, I/O interface 408, and software block 410. These blocks may represent one or more processors or processing circuitries, operating systems, memories, I/O interfaces, applications, and/or software modules. In other implementations, device 400 may not have all of the components shown and/or may have other elements including other types of elements instead of, or in addition to, those shown herein.

In general, a computer that performs the processes described herein (e.g., one or more of the methods of FIGS. 1 and 3) can include one or more processors and a memory (e.g., a non-transitory computer readable medium). The process data and instructions may be stored in the memory. These processes and instructions may also be stored on a storage medium such as a hard drive (HDD) or portable storage medium or may be stored remotely. Note that each of the functions of the described embodiments may be implemented by one or more processors or processing circuits. A processing circuit can include a programmed processor, as a processor includes circuitry. A processing circuit; circuitry may also include devices such as an application specific integrated circuit (ASIC) and conventional circuit components arranged to perform the recited functions. The processing circuitry can be referred to interchangeably as circuitry throughout the disclosure. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device.

The processor may contain one or more processors and even may be implemented using one or more heterogeneous processor systems. According to certain implementations, the instruction set architecture of the processor can use a reduced instruction set architecture, a complex instruction set architecture, a vector processor architecture, a very large instruction word architecture. Furthermore, the processor can be based on the Von Neumann model or the Harvard model. The processor can be a digital signal processor, an FPGA, an ASIC, a PLA, a PLD, or a CPLD. Further, the processor can be an x86 processor by Intel or by AMD; an ARM processor, a Power architecture processor by, e.g., IBM; a SPARC architecture processor by Sun Microsystems or by Oracle; or other known CPU architecture.

The functions and features described herein may also be executed by various distributed components of a system. For example, one or more processors may execute the functions, wherein the processors are distributed across multiple components communicating in a network. The distributed components may include one or more client and server machines, which may share processing in addition to various human interface and communication devices (e.g., display monitors, smart phones, tablets, personal digital assistants (PDAs)). The network may be a private network, such as a LAN or WAN, or may be a public network, such as the Internet. Input to the system may be received via direct user input and received remotely either in real-time or as a batch process. Additionally, some implementations may be performed on modules or hardware not identical to those described. Accordingly, other implementations are within the scope that may be claimed.

### Experimental Evaluation

Tests used 70% of the dataset for training and 30% for testing. To apply an implementation of the disclosed method for selecting the features, a cutoff threshold of the individual techniques Chi-square, IG, RelifF and SVMAttributeEval of zero was used instead of the default threshold (−1.8) to eliminate attributes with ranks of zero or lower.

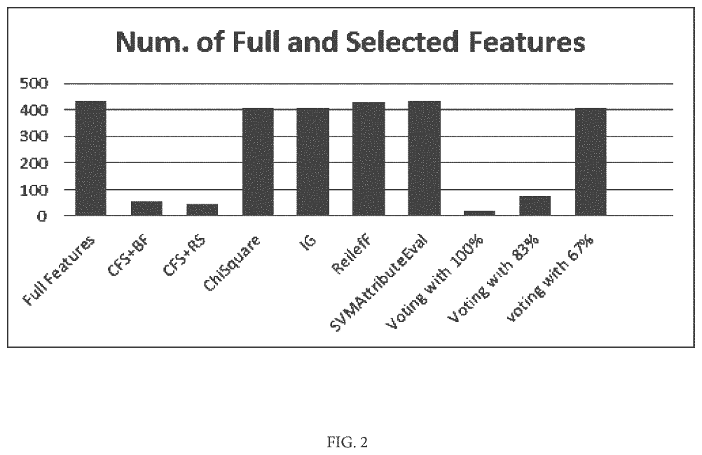

The number of full features, selected features using single techniques and selected features using our proposed technique are shown in FIG. 2. In addition to the full feature vector, three feature vectors called voting 100% FV with 22 features, voting 83% FV with 76 features and voting 67% FV with 411 features were extracted.

### Performance Evaluation Measures

The test approach was evaluated using well-known performance metrics including accuracy, recall, precision, and F-measure.

To calculate these measures a number of true positives (TP), false positives (FP), true negatives (TN), and false negatives (FN) were needed. TN is the number of correctly classified negative samples and TP is the number of correctly classified positive samples. FN is the number positive samples classified incorrectly while FP is the number of negative samples classified incorrectly.

$$Accuracy = \frac{TP + TN}{TP + FP + TN + FN} \times 100$$

$$Recall = \frac{TP}{TP + FN} \times 100$$

$$Precision = \frac{TP}{TP + FP} \times 100$$

$${F}_{1} - measure = \frac{2 * Precision * Recall}{Precision + Recall}$$

### EXAMPLES

Several experiments were conducted using the individual and ensemble classifiers implemented on WEKA (see I. H. Witten and E. Frank, Data Mining: Practical Machine Learning Tools and Techniques. 2005, which is incorporated herein by reference). The first experiment uses the SMO-SVM classifier with PUK kernel using the four feature vectors and the results are shown in Table II. The highest results were obtained using Voting100% FV in terms of precision, recall, F-measure and accuracy which is composed of 22 features.

TABLE II
THE RESULTS USING SMO-SVM USING PUK KERNEL

| | | | F- | |
|---|---|---|---|---|
| | Precision | Recall | Measure | Accuracy |
| Full FV | 35.6 | 52.9 | 40.4 | 52.94 |
| Voting 100% FV | 63.7 | 70.6 | 63.7 | 70.59 |
| Voting 83% FV | 56.4 | 64.7 | 56.9 | 64.71 |
| Voting 67% FV | 35.8 | 52.9 | 40.6 | 52.94 |

Regarding the second experiment, a K-NN classifier was used and the results are shown in Table III. The selected features using the proposed technique perform better than the full features in all cases. The highest results were obtained using Voting 83% FV with 76 features.

TABLE III
THE RESULTS USING K-NN

| | | | F- | |
|---|---|---|---|---|
| | Precision | Recall | Measure | Accuracy |
| Full FV | 77.9 | 64.7 | 64.7 | 64.71 |
| Voting 100% FV | 81.4 | 88.2 | 83.9 | 88.24 |
| Voting 83% FV | 97.1 | 94.1 | 94.7 | 94.12 |
| Voting 67% FV | 83.8 | 70.6 | 70.6 | 70.59 |

The results obtained using an NB classifier are shown in Table IV. The selected features using the proposed technique perform better than the completed features in all cases. The highest results are obtained using Voting 100% FV with 22 features.

TABLE IV
THE RESULTS USING NB

| | | | F- | |
|---|---|---|---|---|
| | Precision | Recall | Measure | Accuracy |
| Full FV | 35.7 | 52.9 | 40.5 | 52.94 |
| Voting 100% FV | 60.3 | 64.7 | 59.2 | 64.71 |
| Voting 83% FV | 44.9 | 58.8 | 48.9 | 58.82 |
| Voting 67% FV | 42.2 | 58.8 | 47.1 | 58.82 |

The previous individual classifiers were combined to build an ensemble classifier using a stacking method. The results are shown in Table V. The selected features using the proposed technique perform better than the completed features in all cases. The highest results are obtained using Voting 100% FV with 22 features.

TABLE V
THE ENSEMBLE CLASSIFIER'S RESULTS

| | | | F- | |
|---|---|---|---|---|
| | Precision | Recall | Measure | Accuracy |
| Full FV | 77.5 | 70.6 | 69.2 | 70.59 |
| Voting 100% FV | 100 | 100 | 100 | 100 |
| Voting 83% FV | 94.1 | 88.2 | 88.8 | 88.24 |
| Voting 67% FV | 83.8 | 70.6 | 68.6 | 70.59 |

As can be seen from TABLE VI, the best results were obtained using the features selected by an implementation of the disclosed feature selection technique in all cases. In addition applying ensemble classification techniques improve the results obtained using the single classifiers in case of SMO, k-NN and NB, significantly.

TABLE VI
THE BEST PERFORMANCE OF EACH CLASSIFIER IN TERM OF PRECISION,
RECALL, F-MEASURE AND THE SIZE OF FEATURE VECTORS

| Classifier | Feature vector | Num. of features | Precision | Recall | F-Measure | Accuracy |
|---|---|---|---|---|---|---|
| SMO-SVM | Voting 100% FV | 22 | 63.7 | 70.6 | 63.7 | 70.59 |
| k-NN | Voting 83% FV | 76 | 97.1 | 94.1 | 94.7 | 94.12 |
| NB | Voting 100% FV | 22 | 60.3 | 64.7 | 59.2 | 64.71 |
| Ensemble classifier | Voting 100% FV | 22 | 100 | 100 | 100 | 100 |

A number of implementations have been described. Nevertheless, it will be understood that various modifications may be made without departing from the spirit and scope of this disclosure. For example, preferable results may be achieved if the steps of the disclosed techniques were performed in a different sequence, if components in the disclosed systems were combined in a different manner, or if the components were replaced or supplemented by other components. The functions, processes and algorithms described herein may be performed in hardware or software executed by hardware, including computer processors and/or programmable circuits configured to execute program code and/or computer instructions to execute the functions, processes and algorithms described herein. Additionally, an implementation may be performed on modules or hardware not identical to those described. Accordingly, other implementations are within the scope that may be claimed.

What is claimed is:

1. A method for detecting the presence of one or more airborne chemical compounds, comprising:
receiving sensor data from one or more chemical sensors;
extracting one or more features from the sensor data;
selecting a group of selected features from among the one or more features;
classifying the group of selected features using one or more models; and
providing an indication of the airborne chemical compounds based on the classifying.

2. The method of claim 1, wherein the extracting includes extracting a portion of time series data from the sensor data.

3. The method of claim 1, wherein selecting the group of selected features includes performing one or more feature selection techniques to determine one or more significant features, wherein the group of selected features includes the one or more significant features.

4. The method of claim 1, wherein the classifying includes performing ensemble classification.

5. The method of claim 1, wherein the selecting includes performing a heterogeneous feature selection technique including one or more of filtering, wrapping, or an embedded technique.

6. The method of claim 1, further comprising:
identifying the concentration of one or more of the airborne chemical compounds in air.

7. A non-transitory computer readable medium having instructions stored therein that, when executed by one or more processors, cause the one or more processors to perform a method comprising:
receiving sensor data from one or more chemical sensors for detecting one or more airborne chemical compounds;
extracting one or more features from the sensor data;
selecting a group of selected features from among the one or more features;
classifying the group of selected features using one or more models; and
providing an indication of sensed material based on the classifying.

8. The non-transitory computer readable medium of claim 7, wherein the extracting includes extracting a portion of time series data from the sensor data.

9. The non-transitory computer readable medium of claim 7, wherein selecting the group of selected features includes performing one or more feature selection techniques to determine one or more significant features, wherein the group of selected features includes the one or more significant features.

10. The non-transitory computer readable medium of claim 7, wherein the classifying includes performing ensemble classification.

11. The non-transitory computer readable medium of claim 7, wherein the selecting includes performing a heterogeneous feature selection technique including one or more of filtering, wrapping, or an embedded technique.

12. The non-transitory computer readable medium of claim 7, wherein the sensed material includes one or more gases and their respective concentrations.

13. A system comprising:
one or more processors coupled to a non-transitory computer readable medium having stored thereon software instructions that, when executed by the one or more processors, cause the one or more processors to perform operations including:
receiving sensor data from one or more chemical sensors for detecting one or more airborne chemical compounds;
extracting one or more features from the sensor data;
selecting a group of selected features from among the one or more features;
classifying the group of selected features using one or more models; and
providing an indication of the one or more airborne chemical compounds based on the classifying.

14. The system of claim 13, wherein the extracting includes extracting a portion of time series data from the sensor data.

15. The system of claim 13, wherein selecting the group of selected features includes performing one or more feature selection techniques to determine one or more significant features, wherein the group of selected features includes the one or more significant features.

16. The system of claim 13, wherein the classifying includes performing ensemble classification.

17. The system of claim 13, wherein the selecting includes performing a heterogeneous feature selection technique including one or more of filtering, wrapping, or an embedded technique.
